(19) United States
(12) Patent Application Publication
(10) Pub. No.: US 2009/0062745 A1
(43) Pub. Date: Mar. 5, 2009

(54) Safety Syringe

(76) Inventor: Can-Long Qiu, Shanghai (CN)

Correspondence Address: LIN & ASSOCIATES INTELLECTUAL PROPERTY, INC., P.O. BOX 2339, SARATOGA, CA, 95070-0339, US

(21) Appl. No.: 11/849,248

(22) Filed: Aug. 31, 2007

(51) Int. Cl. A61M 5/32

(52) U.S. Cl. 604/197; 604/110

(57) ABSTRACT

A safety syringe includes a barrel in which a plunger with a stopper is inserted. A protrusion extends axially from the first end of the plunger and a grab member is engaged with the protrusion. A locking unit is received in the barrel and includes a first tube and a second tube. A needle unit has a needle connected to a first end thereof and a flange extending radially from a second end thereof. The flange is engaged with a groove defined in an inner periphery of the second tube. The grab member is deformed to be pushed into the first tube when the plunger is pushed toward the locking unit, and two ends of the grab member are stopped by an inner periphery of the first tube when the plunger is pulled backward to pull the needle unit, the needle and the locking unit into the barrel.

### BACKGROUND OF THE INVENTION

1. Field of the Invention

The present invention relates generally to a safety syringe which includes a stop plate on a plunger to prevent unintentional connection of the plunger and a locking unit of a needle.

2. The Prior Arts

A conventional safety syringe includes a connection mechanism which allows the plunger to be connected to the locking unit of the needle after injecting the medicine in the barrel from the needle. The locking unit of the needle and the plunger are then firmly connected to each other when the plunger is pulled backward. The needle together with the locking unit is pulled back into the barrel so that the needle does not injure the users or the janitors. Some of the connection mechanism includes a safety ring on the locking unit of the needle, the safety ring includes a plurality of pawls which are able to be expanded outward, and the plunger includes a protrusion which is shaped to penetrate through the safety ring and pushes the pawls which then stop the protrusion from being disengaged therefrom. The protrusion is engaged with the safety ring when the plunger is pushed into the barrel. After injecting the medicine in the barrel, the user pulls the plunger backward to pull the needle and the locking unit into the barrel. However, the safety mechanism cannot avoid the unintentional push of the plunger in the barrel to connect the protrusion and the safety ring. Once the protrusion and the safety ring are connected, they are not able to be separated and the syringe cannot be used anymore. Besides, the needles of the conventional safety syringes are made integrally with the locking unit of the needle so that the needle cannot be replaced and these conventional safety syringes do not meet the practical needs in the market.

### SUMMARY OF THE INVENTION

A primary objective of the present invention is to provide a safety syringe which includes a stop plate on a plunger so as to prevent the user unintentionally pushes the plunger into a barrel to be connected with a locking unit of a needle.

Another objective of the present invention is to provide a safety syringe, which includes a needle unit detachably engaged with the locking unit, such that the needle on the needle unit can be easily replaceable.

The present invention provides a safety syringe comprising a barrel having an open first end, and a plunger is inserted into the barrel. A protrusion extends axially from the first end of the plunger and through a stopper on the first end of the plunger. A slot is defined in the protrusion, and a grab member is engaged with the slot. A needle unit has a needle connected to a first end thereof and a flange extends radially from a second end of the needle unit. A locking unit is received in the barrel and includes a first tube and a second tube. The first tube is located at a center of the second tube with a connection plate connected therebetween. The second tube is engaged with an inner periphery of the barrel and the first tube extends through the open first end of the barrel. A groove is defined in an inner periphery of the second tube, and the flange of the needle unit is engaged with the groove. The grab member is deformed to be pushed into the first tube when the plunger is pushed toward the locking unit, and two ends of the grab member are stopped by an inner periphery of the first tube when the plunger is pulled backward to pull the needle unit, the needle and the locking unit into the barrel.

### BRIEF DESCRIPTION OF THE DRAWINGS

The present invention will be apparent to those skilled in the art by reading the following detailed description of a preferred embodiment thereof, with reference to the attached drawings, in which:

FIG. 1A is a cross sectional view to show a needle unit of a safety syringe in accordance with the present invention;

FIG. 1B is a perspective view of the needle unit of the safety syringe in accordance with the present invention;

FIG. 2A is a cross sectional view to show a locking unit of the safety syringe in accordance with the present invention;

FIG. 2B is a perspective view of the locking unit of the safety syringe in accordance with the present invention;

FIG. 3A is a cross sectional view to show a barrel of the safety syringe in accordance with the present invention;

FIG. 3B is a perspective view of the barrel of the safety syringe in accordance with the present invention;

FIG. 4A is a cross sectional view to show a plunger of the safety syringe in accordance with the present invention;

FIG. 4B is a perspective view of the plunger of the safety syringe in accordance with the present invention;

FIG. 5A is a cross sectional view to show that the plunger is pushing into the barrel;

FIG. 5B is a cross sectional view to show that the stop plate is pushed inward so that the plunger can be completely pushed into the barrel;

FIG. 5C is a cross sectional view to show that the plunger is pulled back and broken to accommodate the needle, the needle unit and the locking unit in the barrel; and

FIG. 6 is an exploded view of the safety syringe of the present invention.

### DETAILED DESCRIPTION OF THE PREFERRED EMBODIMENT

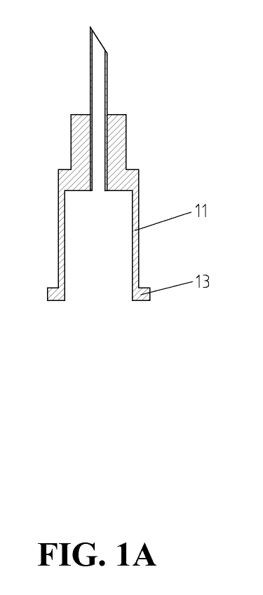

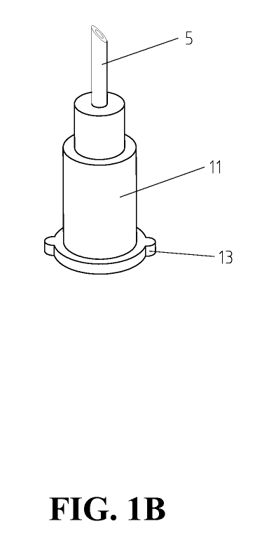

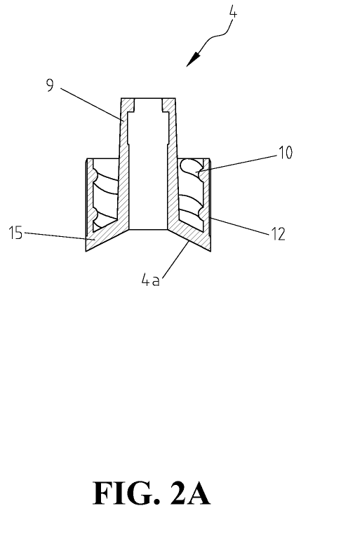

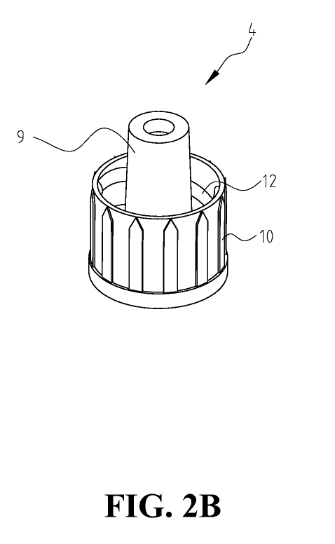

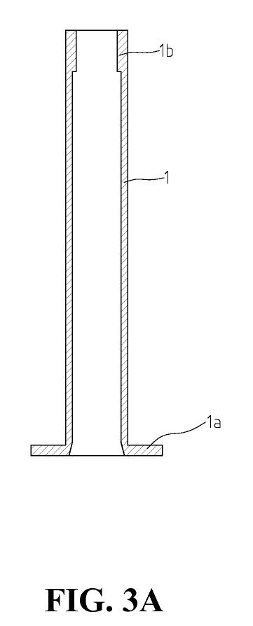

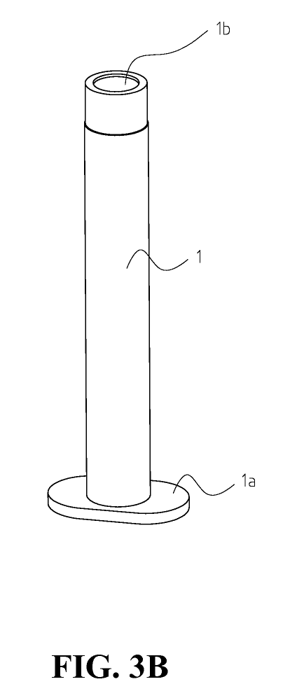

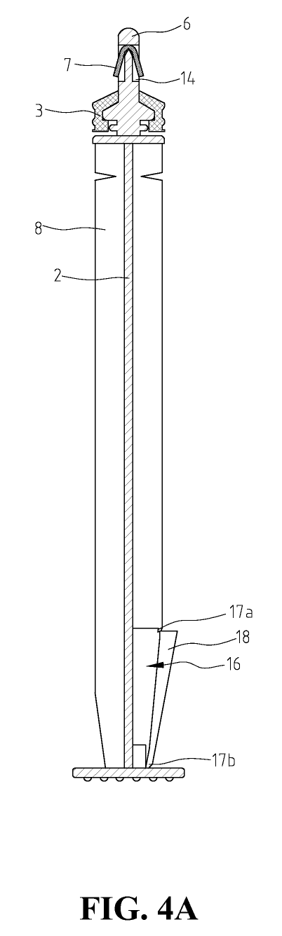

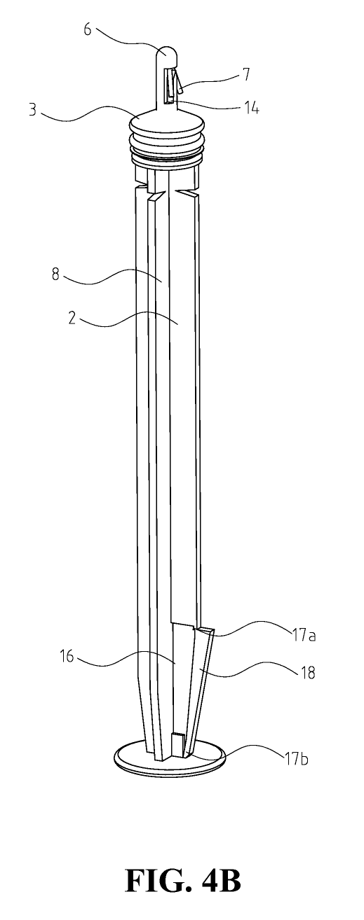

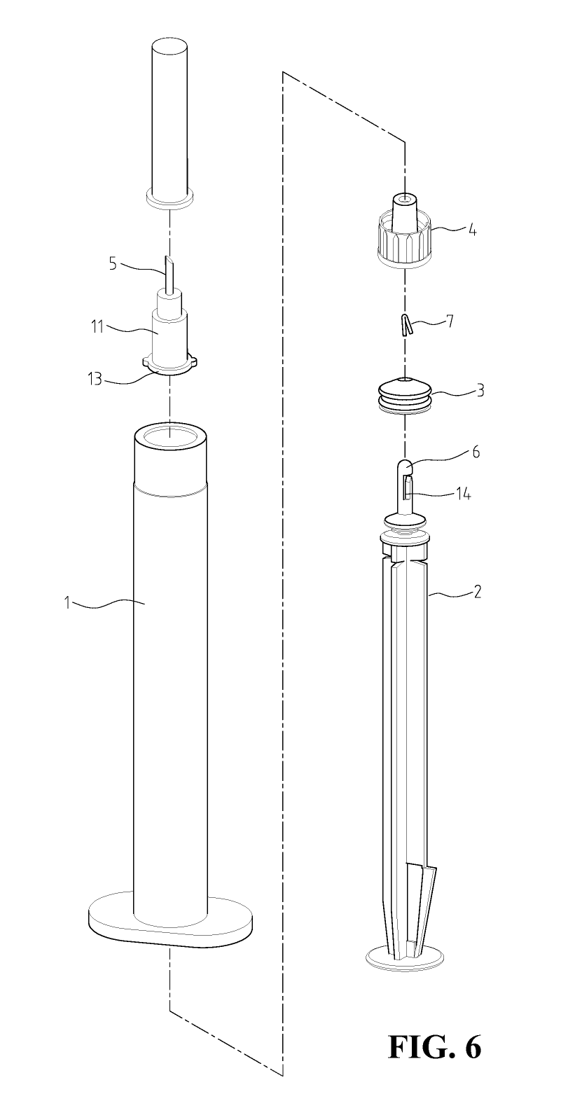

With reference to the drawings and in particular to FIGS. 1A, 1B, 2A, 2B, 3A, 3B, 4A, 4B and 6, a safety syringe in accordance with the present invention comprises a barrel 1 which has an open first end and an open second end, a plurality of stop ridges 1b extend inward from an inner periphery of the open first end of the barrel 1, and a finger flange 1a extends radially from the open second end of the barrel 1.

A plunger 2 has a first one end thereof inserted into the barrel 1 from the open second end of the barrel 1, and a stopper 3 is connected to the first end of the plunger 2. A protrusion 6 extends axially from the first end of the plunger 2 and through the stopper 3. A slot 14 is defined in the protrusion 6, and a grab member 7 is engaged with the slot 14. The grab member 7 is made by metallic material and can be substantially a straight member, a U-shaped member or a reversed U-shaped member. The grab member 7 is deformable when being pressed. The plunger 2 further includes a plurality of ribs 8 and one of the ribs 8 includes an opening 16. A thumb rest 2a is connected to a second end of the plunger 2. A stop plate 18 has one end connected to an outer corner of the rib 8 having the opening 16 at a first connection point 17a and the other end of the stop plate 18 is connected to the thumb rest 2a at a second connection point 18a. The stop plate 18 protrudes beyond an outer side of the rib 8 having the opening 16 so as to be stopped by the finger flange 1a on the second end of the barrel 1, such that the plunger 2 cannot be unintentionally pushed into the barrel 1 completely.

A needle unit 11 includes a needle 5 connected to a first end thereof, and a flange 13 extends radially from a second end of the needle unit 11. A locking unit 4 is received in the barrel 1 and includes a first tube 9 and a second tube 10 which is engaged with the stop ridges 1b of the barrel 1. The first tube 9 is located at a center of the second tube 10 with a connection plate 4a connected between two respective first open ends of the first tube 9 and second tubes 10. A diameter of the second tube 10 is larger than a diameter of the first tube 9. The first tube 9 extends through the open first end of the barrel 1 and defines an annular space between the first tube 9 and the inner periphery of the open first end of the barrel 1. A groove 12 is defined in an inner periphery of the second tube 10. The needle unit 11 is mounted to the first tube 9 and the flange 13 is rotatably engaged with the groove 12 of the second tube 10 via a second open end of the second tube 10.

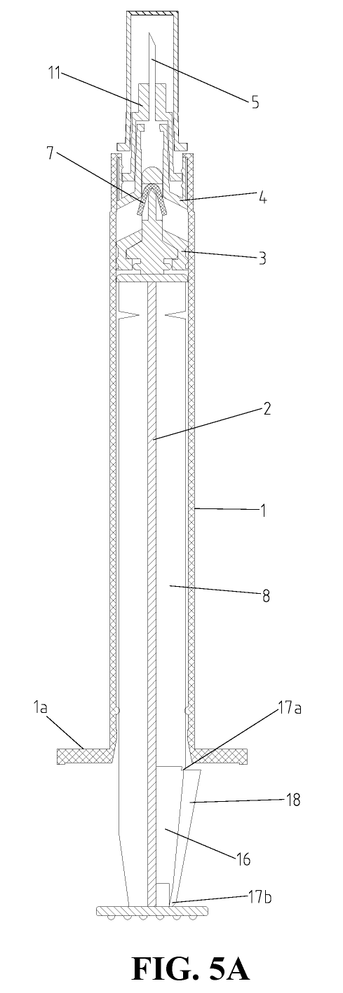

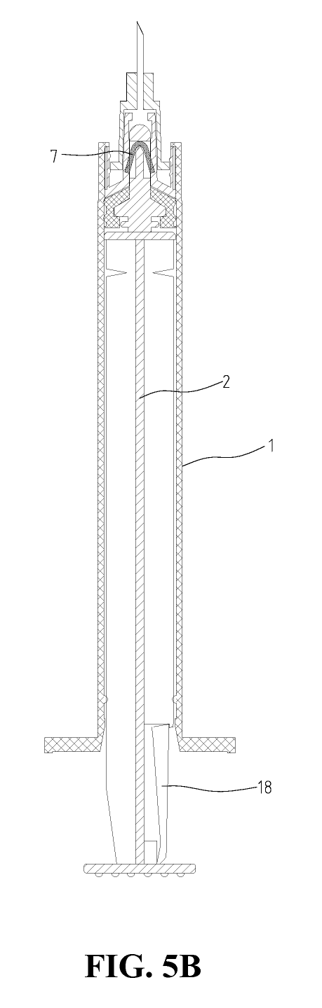

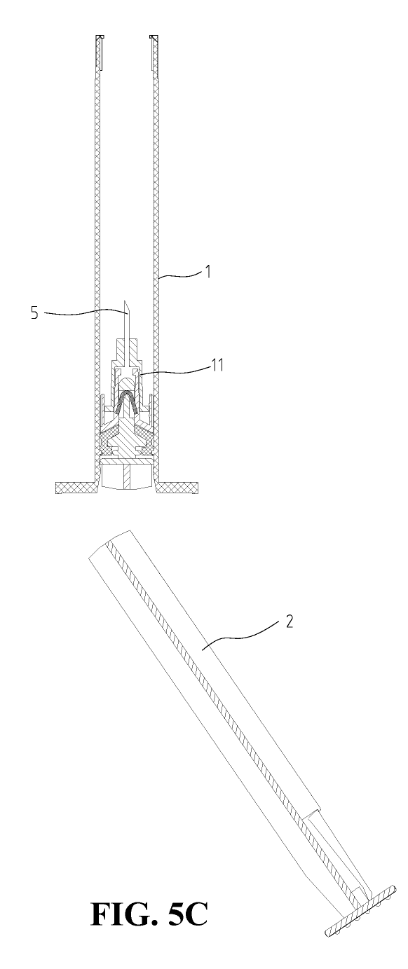

As shown in FIGS. 5A to 5C, when the user uses the safety syringe, the plunger 2 is inserted into the barrel 1 and the stop plate 18 is stopped by the finger flange 1a so that the protrusion 6 and the grab member 7 on the first open end of the plunger 2 is restricted not to inserted into the first tube 9 of the locking unit 4. Therefore, the grab member 7 is not deformed and connected to the first tube 9. The needle 5 is inserted to a medicine container, and the plunger 2 is pulled backward to suck the medicine into the barrel 1. When proceeding injection, the plunger 2 is pushed into the barrel 1 and the stop plate 18 is pushed inward to break the first connection points 17a such that the stop plate 18 is accommodated in the opening 16. The plunger 2 then can be completely pushed into the barrel 1 to eject the medicine in the barrel 1 out from the needle 5. The protrusion 6 together with the grab member 7 is inserted into the first tube 9. The grab member 7 is bent when it is inserted into the first tube 9, and two ends of the grab member 7 are stopped by the inner periphery of the first tube 9 to firmly connect the grab member 7 with the first tube 9. After the injection, the plunger 2 is pulled backward, and the needle unit 11, the needle 5 and the locking unit 4 are pulled into the barrel 1. The ribs 8 of the plunger 2 each include a notch located close to the first end thereof such that the plunger 2 can be easily broken at the notches as shown in FIG. 5C.

Although the present invention has been described with reference to the preferred embodiment thereof, it is apparent to those skilled in the art that a variety of modifications and changes may be made without departing from the scope of the present invention which is intended to be defined by the appended claims.

What is claimed is:

1. A safety syringe comprising
a barrel having an open first end;
a plunger having a first one end thereof inserted into the barrel, a stopper connected to the first end of the plunger, a protrusion extending axially from the first end of the plunger and through the stopper, a slot defined in the protrusion, and a grab member engaged with the slot;
a needle unit having a needle connected to a first end thereof and a flange extending radially from a second end of the needle unit; and
a locking unit received in the barrel and including a first tube and a second tube, the first tube located at a center of the second tube with a connection plate connected therebetween, a diameter of the second tube being larger than a diameter of the first tube, the first tube extending through the open first end of the barrel, a groove defined in an inner periphery of the second tube, and the flange of the needle unit being engaged with the groove,
wherein the grab member is deformed to be pushed into the first tube when the plunger is pushed toward the locking unit, and two ends of the grab member are stopped by an inner periphery of the first tube when the plunger is pulled backward to pull the needle unit, the needle and the locking unit into the barrel.

2. The safety syringe as claimed in claim 1, wherein the connection plate is connected between two respective first open ends of the first tube and second tube, the needle unit is mounted to the first tube, and the flange of the needle unit is rotatably engaged with the groove of the second tube via a second open end of the second tube.

3. The safety syringe as claimed in claim 1, wherein the plunger includes a plurality of ribs and one of the ribs includes an opening, a thumb rest is connected to a second end of the plunger, a stop plate has one end connected to an outer corner of the rib having the opening at a first connection point and the other end of the stop plate is connected to the thumb rest at a second connection point, the stop plate protrudes beyond an outer side of the rib having the opening so as to be stopped by a finger flange on a second end of the barrel.

4. The safety syringe as claimed in claim 3, wherein the opening is sized to accommodate the stop plate which is pushed to break the first connection points.

5. The safety syringe as claimed in claim 1, wherein the grab member is made by metallic material, and is substantially a straight member, a U-shaped member or a reversed U-shaped member.

6. The safety syringe as claimed in claim 3, wherein the grab member does not enter into the first tube when the stop plate is stopped by the finger flange.
